(19) United States
(12) Patent Application Publication
(10) Pub. No.: US 2026/0057973 A1
(43) Pub. Date: Feb. 26, 2026

(54) METHOD AND APPARATUS FOR ESTIMATING PHYSICAL PROPERTIES OF A MATERIAL FROM CRYSTAL STRUCTURE DATA

(71) Applicant: AISTAR CO., LTD, Seoul (KR)

(72) Inventors: Woo Jung JANG, Seoul (KR); Woo Young JEONG, Seoul (KR)

(73) Assignee: AISTAR CO., LTD, Seoul (KR)

(21) Appl. No.: 19/304,573

(22) Filed: Aug. 19, 2025

(30) Foreign Application Priority Data
Aug. 20, 2024 (KR) 10-2024-0111347

(51) Int. Cl. G16C 60/00; G16C 20/30; G16C 20/70; G16C 20/90

(52) U.S. Cl. CPC G16C 60/00; G16C 20/30; G16C 20/70; G16C 20/90

(57) ABSTRACT

The present disclosure relates to materials science using processing of crystallographic structure data and artificial intelligence, and more particularly to methods and apparatuses for estimating material properties from crystallographic descriptive data, wherein a computer-implemented method includes generating first data representing a crystal structure from crystallographic descriptive data; generating, in view of structural periodicity, second data representing an expanded supercell; converting the second data into input data as a four-dimensional tensor in which a first dimension corresponds to atom species and remaining dimensions correspond to coordinates of a discretized three-dimensional grid; supplying the input data to a neural network including convolutional layers and a self-attention mechanism to extract features; and estimating, from the extracted features, at least one material property of the material. Related apparatuses and non-transitory computer-readable media are also disclosed.

### CROSS-REFERENCE TO RELATED APPLICATIONS

This application claims priority to and the benefit of Korean Patent Application No. 10-2024-0111347, filed on Aug. 20, 2024, the disclosure of which is incorporated by reference herein in its entirety.

### BACKGROUND

The present disclosure relates to the field of materials science utilizing processing of crystallographic structure data and artificial intelligence. More particularly, the disclosure pertains to methods and apparatuses for estimating physical properties of materials from crystal-structure descriptive data.

Crystal structure plays a pivotal role in elucidating fundamental characteristics of matter. Consequently, analysis of crystal structures is central to materials science because the arrangement of atoms or molecules strongly influences electrical, mechanical, and thermal properties. Accurate understanding and analysis of crystal structure is essential to develop new materials and to improve performance of existing materials.

Traditionally, structure analysis has relied on experimental techniques such as X-ray diffraction (XRD) and neutron diffraction. While highly accurate, these approaches involve complex sample preparation, measurements, and data interpretation, require expensive instruments and specialized expertise, and thus incur significant time and cost.

Computer simulations, e.g., molecular dynamics (MD) and density functional theory (DFT), have attracted interest as alternatives that model atomic-level interactions to predict structures. However, these methods can be computationally expensive and time-consuming, especially for large systems or complex crystals.

Against this backdrop, machine learning and artificial intelligence (AI) have emerged as promising alternatives. Machine-learning models excel at learning patterns from large datasets and predicting unseen data. Deep learning techniques such as convolutional neural networks (CNNs) are well-suited to images and 3D structural data. CNNs learn local patterns through layered architectures and can infer global structure therefrom. Attention mechanisms further enhance performance by enabling a model to focus on salient parts of the input via learned weights, improving predictions for complex structures and diverse materials.

The foregoing background is presented to provide context and is not to be construed as an admission that any item discussed herein was publicly known or forms part of the prior art as of any relevant date.

### SUMMARY

Embodiments of the present disclosure provide methods and apparatuses that efficiently process crystal-structure data and, based thereon, estimate material properties rapidly and accurately. In particular, embodiments generate a four-dimensional (4D) tensor from crystallographic data and input the tensor to a neural network to predict material properties.

In one aspect, a computer-implemented method includes: generating first data indicating a crystal structure from crystallographic descriptive data; based on the first data and the periodicity of the crystal structure, generating second data indicating an expanded supercell; converting the second data into input data in a 4D tensor format in which a first dimension corresponds to atom species and three remaining dimensions correspond to 3D spatial coordinates; inputting the input data into a neural network based on convolution and an attention mechanism to extract features; and estimating a material property from the extracted features.

In certain embodiments, the crystallographic descriptive data is in a Crystallographic Information File (CIF) format. Generating the first data may include parsing unit-cell parameters, symmetry operations, and atomic coordinates from the CIF; constructing unit-cell basis vectors; applying symmetry operations to obtain symmetry-equivalent points; and computing atomic positions in the unit cell to produce unit-cell structure data (the first data).

Generating the second data may include determining translation vectors for a user-specified supercell size; translationally replicating the unit cell to construct the supercell; and merging identical atoms at boundaries between adjacent unit cells to produce supercell structure data (the second data).

Converting to input data may include assigning a unique integer identifier to each atom according to species; determining a minimum axis-aligned rectangular parallelepiped enclosing the supercell; dividing its edge lengths by a user-specified resolution to define a 3D grid; identifying, for each grid point, whether an atom is present based on a distance threshold; assigning the atom's identifier or zero to each grid point; converting the 3D grid to a 4D tensor; and supplying the tensor to the neural network. The integer identifier may correspond to atomic number or the periodic-table order. Grid resolution may be user-determined. The neural network may include multiple convolutional layers, pooling layers, and self-attention modules. The property may include at least one of activation energy, Young's modulus, and interfacial energy. The materials may include at least one of a cathode active material, an anode active material, an electrolyte, and a separator for a lithium-metal secondary battery.

In another aspect, a non-transitory computer-readable medium (NCRM) stores instructions that, when executed, cause one or more processors to receive a 4D tensor including 3D atomic coordinate information of a material's crystal structure, input the 4D tensor to a 3D CNN including 3D self-attention modules that compute Q/K/V, attention weights, and weighted feature maps, and predict a material property from extracted features.

According to embodiments, a supercell is generated from crystallographic data, converted into a 4D tensor, and input to a neural network, enabling rapid and accurate estimation of material properties. Compared with purely experimental approaches, time and cost can be reduced while improving prediction accuracy across diverse materials. The effects and benefits are not limited to those expressly mentioned herein.

### BRIEF DESCRIPTION OF THE DRAWINGS

FIG. 1 is a schematic view of a system according to an embodiment.

FIG. 2 is a flowchart schematically illustrating a property-estimation operation of a computer apparatus.

FIG. 3 is a flowchart schematically illustrating generation of first data by the computer apparatus.

FIG. 4 is a flowchart schematically illustrating generation of second data by the computer apparatus.

FIG. 5 is a view illustrating input data according to an embodiment.

FIG. 6 is a diagram illustrating a training operation of the computer apparatus.

FIG. 7 is a block diagram schematizing a neural network model.

FIG. 8 is a table showing property-estimation elapsed time.

FIG. 9 is a block diagram of a computer apparatus.

### DETAILED DESCRIPTION

The terms used herein are used merely to describe specific embodiments and are not intended to limit the scope of other embodiments. A singular expression may include a plural expression unless the context clearly indicates otherwise. Technical or scientific terms used herein may have the same meaning as commonly understood by one of ordinary skill in the art to which this disclosure belongs. Terms used herein, including those defined in general dictionaries, may be interpreted as having a meaning that is the same as or similar to the meaning in the context of the relevant art and are not to be interpreted in an idealized or overly formal sense unless expressly so defined herein. In some cases, even terms defined herein should not be interpreted to exclude embodiments of the present disclosure.

Hereinafter, various embodiments will be described in detail with reference to the accompanying drawings so that those skilled in the art can easily implement them. However, the technical idea of the present disclosure can be modified and implemented in various forms and is not limited to the embodiments described herein. In describing the embodiments disclosed in this specification, if it is determined that a detailed description of related known technology may obscure the gist of the technical idea, a detailed description thereof will be omitted. Identical or similar constituent elements are assigned the same reference numerals, and redundant descriptions thereof will be omitted.

Herein, the term “-unit” or “-module” refers to a component that performs a specific function, implemented by software or hardware such as a Field Programmable Gate Array (FPGA) or an Application Specific Integrated Circuit (ASIC). However, “-unit” or “-module” is not limited to being performed by software or hardware. A “-unit” or “-module” may exist in the form of data stored in an addressable storage medium and may be configured to cause one or more processors to execute a specific function as implemented by instructions.

Software may include a computer program, code, instructions, or one or more combinations thereof, and may configure a processing device to operate as desired or may command the processing device independently or collectively. Software and/or data may be permanently or temporarily embodied in any type of machine, component, physical device, virtual equipment, computer storage medium or device, or transmitted signal wave to be interpreted by or to provide instructions or data to a processing device. Software may be distributed over networked computer systems and may be stored or executed in a distributed manner. Software and data may be stored on one or more computer-readable recording media. The software may be read into a main memory from another computer-readable medium, such as a data storage device, or from another device via a communication interface. Software instructions stored in the main memory may cause a processor to perform processes or steps described in detail below. Alternatively, processes consistent with the principles of the present disclosure may be implemented using hardwired circuitry instead of or in combination with software instructions. Thus, embodiments consistent with the principles of the present disclosure are not limited to any specific combination of hardware circuitry and software.

The terminology used in this application is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises,” “comprising,” “includes,” and/or “having,” when used in this specification, specify the presence of stated features, integers, steps, operations, elements, and/or components, but do not preclude the presence or addition of one or more other features, integers, steps, operations, elements, components, and/or groups thereof. The terms “first,” “second,” etc., may be used to describe various components, but the components should not be limited by these terms. These terms are only used to distinguish one component from another.

As used herein, “learning” refers to the process by which an artificial neural network extracts and internalizes useful patterns or features from data. During the learning process, the neural network adjusts its internal parameters to model the relationship between a given input and the expected output. This is primarily achieved by updating the parameters in a direction that minimizes a loss function, and an optimization algorithm such as gradient descent may generally be used. The learning process is typically conducted over multiple epochs, and in each epoch, the entire training dataset may be fed into the neural network once.

As used herein, “neural network model” may include any form of algorithm or methodology used to learn or understand specific patterns or structures from data. That is, a neural network model can include not only machine learning models such as regression models, decision trees, random forests, support vector machines, K-nearest neighbors, naive Bayes, and clustering algorithms, but also deep learning models such as neural networks, convolutional neural networks, recurrent neural networks, Transformer-based neural networks, Generative Adversarial Networks (GANs), and autoencoders. A “neural network model” may refer to a set of learned parameters or weights used to predict or classify an output for a specific input, and this model can be trained through methods such as supervised learning, unsupervised learning, semi-supervised learning, and reinforcement learning. Furthermore, it may include not only a single model but also various learning methods and structures such as ensemble models, multi-modal models, and models based on transfer learning. Such a neural network model may be pre-trained on a separate computer device from the computer device that predicts the output for an input, and then used on the other computer device. For example, the neural network model in the present disclosure may include a 3D Convolutional Neural Network and a Self-Attention Mechanism, which can effectively process 3D crystal structure data.

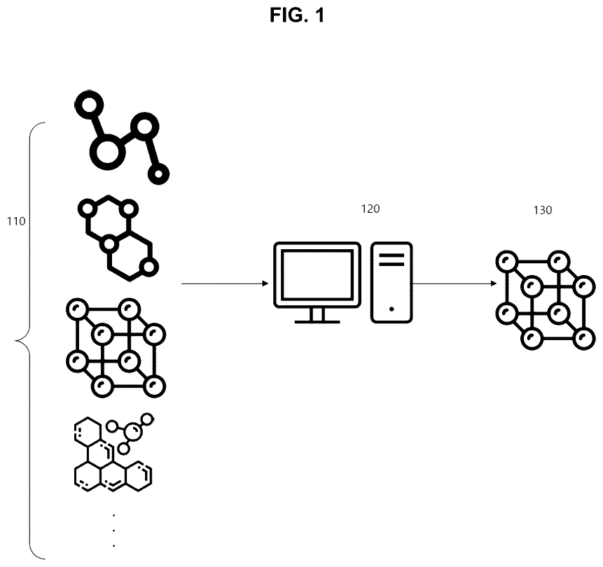

FIG. 1 is a diagram schematically illustrating a system according to an embodiment of the present disclosure. FIG. 1 shows a system according to an embodiment. The system may refer to a system that inputs crystal structure description data 110 corresponding to a plurality of materials into a computer device 120 to estimate the properties of each material and select a material suitable for the properties. The system according to an embodiment may be a system for selecting a suitable material for a lithium-metal secondary battery cell from crystal structure description data 110.

A computer device 120 according to an embodiment may perform a series of processes to predict material properties from crystal structure description data.

The computer device 120 according to an embodiment may generate first data indicating a crystal structure from the crystal structure description data. This crystal structure description data may be in the Crystallographic Information File (CIF) format. The computer device 120 may generate second data indicating an expanded supercell that considers the periodicity of the crystal structure, based on the first data.

The computer device 120 according to an embodiment may convert the second data into input data in the form of a four-dimensional tensor. This four-dimensional tensor may have a format where the first dimension represents the species of atoms, and three additional dimensions represent three-dimensional spatial coordinates.

The computer device 120 according to an embodiment may input the input data into a neural network model based on a convolutional neural network and an attention mechanism to extract features. The computer device 120 according to an embodiment may estimate material properties from the extracted features.

The computer device 120 according to an embodiment may also train the neural network model using the input data as features and the properties as labels.

Hereinafter, the operation of the computer device 120 will be described in more detail.

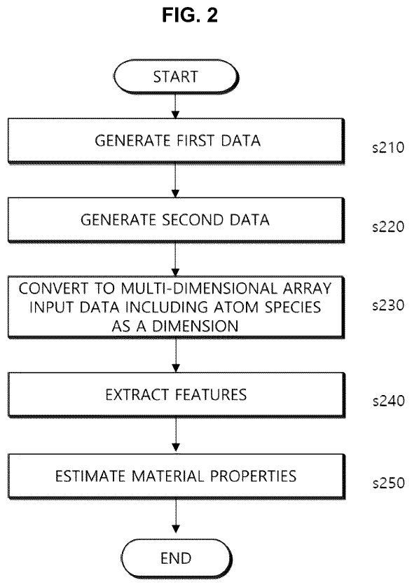

FIG. 2 is a flowchart schematically illustrating an operation for estimating material properties by a computer device according to an embodiment of the present disclosure. Referring to FIG. 2, in step S210, the computer device may generate first data indicating a crystal structure from crystal structure description data.

The computer device may generate first data indicating a crystal structure from the crystal structure description data. The crystal structure description data may be in the Crystallographic Information File (CIF) format.

More specifically, the computer device can parse the unit cell parameters of the crystal structure from the CIF file. Unit cell parameters are variables that define the size and shape of the unit cell, which is the basic repeating unit of a crystal structure, and are generally represented by a, b, c (the lengths of each axis) and a, P, y (the angles between each axis). The computer device can parse the symmetry operations of the crystal structure from the CIF file. Symmetry operations are a set of transformation rules that describe how atoms are arranged within the crystal, expressed as a crystallographic space group, and may include operations such as rotation, reflection, and translation. The computer device can parse the species and coordinates of each atom within the crystal structure from the CIF file. This information is usually contained within a loop_block starting with “atom_site” in the CIF. The parsed unit cell parameters can be used to construct the basis vectors of the unit cell. The basis vectors are vectors that form the edges of the unit cell, generally denoted as a, b, and c. The computer device can apply the parsed symmetry operations to the basis vectors of the unit cell to generate symmetry-equivalent points within the unit cell. Symmetry-equivalent points may indicate points that have the same environment as the original point due to a symmetry operation. The computer device can calculate all atomic positions within the unit cell using the parsed atomic coordinates and the generated symmetry-equivalent points to generate unit cell structure data. This generated unit cell structure data may be the first data. Therefore, the computer device can receive crystal structure description data in CIF format, parse unit cell parameters, symmetry operations, and atomic coordinates, and use these to calculate the basis vectors and symmetry-equivalent points of the unit cell, thereby finally generating the first data, which is unit cell structure data that fully describes the crystal structure.

In step S220, the computer device according to an embodiment may generate second data indicating an expanded supercell that considers the periodicity of the crystal structure, based on the first data. The computer device can determine translation vectors for constructing a supercell of a user-specified size from the first data. Translation vectors are vectors used to build a supercell by repeatedly translating the unit cell by a given direction and distance. For example, if a user specifies a 2×2×2 supercell, the computer device can generate translation vectors by doubling each basis vector of the unit cell. The computer device can construct a supercell composed of a plurality of unit cells by translationally moving the unit cell of the first data using the translation vectors. This is achieved by repeatedly translating the unit cell by the direction and magnitude of the translation vectors to generate adjacent unit cells. For example, in the case of a 2×2×2 supercell, a total of 8 unit cells can be generated by translating the unit cell twice along each basis vector direction. The computer device can generate supercell structure data by merging identical atoms existing at the boundaries between adjacent unit cells within the supercell. The plurality of unit cells generated by translational movement share boundaries, and identical atoms may exist redundantly on these boundaries. The computer device can identify these duplicate atoms and merge them into one, thereby generating the second data, which is continuous and periodic supercell structure data.

In step S230, the computer device according to an embodiment may convert the second data into input data in the form of a four-dimensional tensor, where the first dimension represents the species of atoms and three additional dimensions represent three-dimensional spatial coordinates.

The computer device can assign a unique integer identifier to each atom included in the second data according to the species of the atoms. This integer identifier may correspond to the atomic number or the order of the atom in the periodic table. The computer device can determine the size of a minimum-sized rectangular parallelepiped that encloses the region occupied by the supercell in three-dimensional space. The computer device can generate a three-dimensional grid by dividing the length of each edge of this rectangular parallelepiped by a user-specified resolution. The computer device can identify the presence or absence of an atom at each grid point of the three-dimensional grid. To do this, the computer device can calculate the distance between a grid point and each atom in the second data, and if the minimum distance is less than or equal to a user-specified threshold, it can determine that an atom exists at that grid point. The computer device can construct a three-dimensional grid by assigning the integer identifier of the corresponding atom to grid points where an atom is identified, and 0 to grid points where no atom is present. The computer device can convert this constructed three-dimensional grid into a four-dimensional tensor. In this case, the first dimension of the four-dimensional tensor corresponds to the atom species, and the remaining three dimensions correspond to the three-dimensional spatial coordinates. The computer device can use this four-dimensional tensor as the input data for the neural network model.

In step S240, the computer device according to an embodiment may input the input data into a neural network model based on a convolutional neural network and an attention mechanism to extract features.

The neural network model may be composed of a plurality of convolutional layers, pooling layers, and a self-attention module. Specifically, the input part of the neural network model is equipped with a plurality of 3D convolutional layers that accept data in a four-dimensional tensor format, and between each convolutional layer, an activation function, a batch normalization layer, and a 3D pooling layer may be placed.

In the convolutional layer, a feature map is extracted by applying a 3D convolutional filter to the four-dimensional tensor. The size and number of convolutional filters, as well as the stride, can be set by the user as hyperparameters. The feature map that has passed through the convolutional layer is input into a self-attention module to identify long-range dependencies between features. The self-attention module calculates query, key, and value vectors from the input features and generates an attention map to emphasize important features or suppress unnecessary ones.

The features that have passed through the self-attention module are summarized into a fixed-length vector through pooling, and this vector is used as input to a fully connected layer for property prediction. The computer device can effectively extract features that consider the periodicity of the 3D structure and the interactions between atoms using a neural network model configured in this way.

In step S250, the computer device according to an embodiment estimates material properties such as activation energy, Young's modulus, and interfacial energy from the features extracted by the neural network model. The computer device can estimate the material properties from the features extracted by the neural network model in step S240.

The computer device can predict the material property values based on the extracted features using a regression model or a classification model connected to the output layer of the neural network model. In the case of a regression model, it predicts continuous values such as activation energy and Young's modulus, while a classification model can divide the range of property values into several intervals and predict the corresponding interval. The computer device can evaluate the accuracy of the predicted property values based on the loss function used when training the neural network model. For example, for a regression model, Mean Squared Error or Mean Absolute Error can be used, and for a classification model, a Cross-Entropy loss function can be used.

The computer device can evaluate the generalization performance of the trained model using a validation dataset used during the training of the neural network model. This can confirm whether the model is robust in real applications and not just overfitted to the training data. The computer device can provide the prediction results of the neural network model to the user. This can be visualized through a graphical user interface or provided by being saved in a file format. In addition, a confidence indicator, such as a probability value or an uncertainty measure, can be provided along with the prediction results to allow the user to judge the reliability of the prediction results. The computer device can use the prediction results of the neural network model as input for other calculation models or simulations. For example, the prediction results of the neural network model can be utilized to search for an initial structure in Density Functional Theory (DFT) calculations. This can increase the efficiency of computationally expensive DFT calculations.

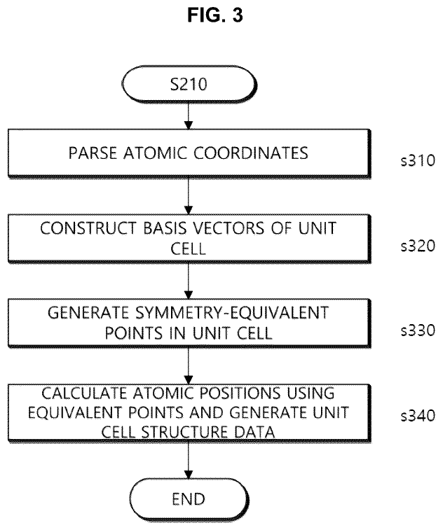

FIG. 3 is a flowchart schematically illustrating an operation for generating first data by a computer device according to an embodiment of the present disclosure.

TABLE 1

# generated using pymatgen
data_Li(WO2)2
_symmetry_space_group_name_H-M ‘P 1’
_cell_length_a 6.36842287
_cell_length_b 6.36842287
_cell_length_c 6.36842287
_cell_angle_alpha 60.00000000
_cell_angle_beta 60.00000000
_cell_angle_gamma 60.00000000
_symmetry_Int_Tables_number 1
_chemical_formula_structural Li(WO2)2
_chemical_formula_sum ‘Li2 W4 O8’
_cell_volume 182.63360132
_cell_formula_units_Z 2
loop_{—}
_symmetry_equiv_pos_site_id
_symmetry_equiv_pos_as_xyz
1 ‘x, y, z’
loop_{—}
_atom_site_type_symbol
_atom_site_label
_atom_site_symmetry_multiplicity
_atom_site_fract_x
_atom_site_fract_y
_atom_site_fract_z
_atom_site_occupancy
Li Li0 1 0.75000000 0.75000000 0.75000000 1
Li Li1 1 0.50000000 0.50000000 0.50000000 1
W W2 1 0.12500000 0.12500000 0.12500000 1
W W3 1 0.12500000 0.12500000 0.62500000 1
W W4 1 0.12500000 0.62500000 0.12500000 1
W W5 1 0.62500000 0.12500000 0.12500000 1
O O6 1 0.91109400 0.36296900 0.36296900 1
O O7 1 0.36296900 0.36296900 0.36296900 1
O O8 1 0.33890600 0.88703100 0.88703100 1
O O9 1 0.88703100 0.88703100 0.33890600 1
O O10 1 0.88703100 0.88703100 0.88703100 1
O O11 1 0.88703100 0.33890600 0.88703100 1
O O12 1 0.36296900 0.36296900 0.91109400 1
O O13 1 0.36296900 0.91109400 0.36296900 1

Table 1 is an example of crystal structure description data, a CIF. Referring to FIG. 3, in step S310, the computer device may parse the unit cell parameters, symmetry operations, and atomic coordinates of the crystal structure from the CIF.

The unit cell parameters are indicated by keywords such as ‘_cell_length_a’, ‘_cell_length_b’, ‘_cell_length_c’, ‘_cell_angle_alpha’, ‘_cell_angle_beta’, and ‘_cell_angle_gamma’, representing the lengths of the unit cell in the width, depth, and height directions and the angles between each axis, respectively. For example, looking at Table 1, ‘_cell_length_a’, ‘_cell_length_b’, and ‘_cell_length_c’ are all indicated as 6.36842287, and ‘_cell_angle_alpha’, ‘_cell_angle_beta’, and ‘_cell_angle_gamma’ are all 60.00000000. The symmetry operations are listed under the keyword ‘_symmetry_equiv_pos_as_xyz’. In the given CIF, there is only one symmetry operation represented as ‘x, y, z’. The atomic coordinates are indicated by keywords such as ‘_atom_site_label’, ‘_atom_site_fract_x’, ‘_atom_site_fract_y’, and ‘atom_site_fract_z’ under the ‘loop’ keyword. These represent the label of the atom and its fractional coordinates within the unit cell, respectively. For example, ‘Li Li0 1 0.75000000 0.75000000 0.75000000 1’ may indicate that the Li atom labeled Li0 is located at the position (0.75, 0.75, 0.75) within the unit cell.

In step S320, the computer device according to an embodiment may construct the basis vectors of the unit cell from the parsed unit cell parameters. The computer device can construct the basis vectors of the unit cell from the parsed unit cell parameters. The basis vectors are three vectors a, b, and c that form the edges of the unit cell, and their magnitudes and directions are calculated from ‘_cell_length_a’, ‘_cell_length_b’, ‘_cell_length_c’ and ‘_cell_angle_alpha’, ‘_cell_angle_beta’, ‘_cell_angle_gamma’. As in Table 1, a, b, and c become vectors with a magnitude of 6.36842287 and an angle of 60 degrees between them.

In step S330, the computer device according to an embodiment may apply the parsed symmetry operations to the basis vectors to generate symmetry-equivalent points within the unit cell. Symmetry-equivalent points are points that have the same environment as the original point due to a symmetry operation. Since there is only one symmetry operation ‘x, y, z’ in the given CIF, no additional equivalent points may be generated in this step.

In step S340, the computer device according to an embodiment may calculate the atomic positions within the unit cell using the parsed atomic coordinates and the generated symmetry-equivalent points to generate unit cell structure data. This includes the species of each atom and its absolute coordinates within the unit cell. The absolute coordinates are obtained by multiplying the fractional coordinates by the basis vectors. For example, for the Li0 atom, the absolute coordinates could be (0.75a, 0.75b, 0.75c).

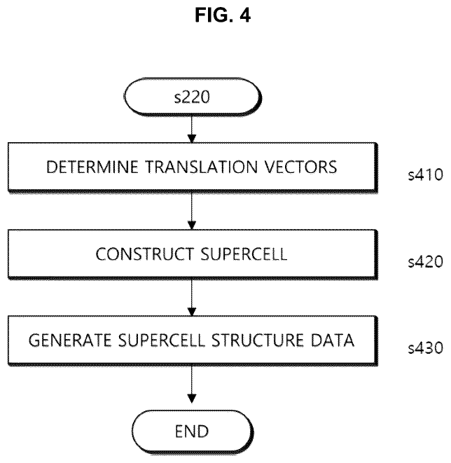

FIG. 4 is a flowchart schematically illustrating an operation for generating second data by a computer device according to an embodiment of the present disclosure.

Referring to FIG. 4, in step S410, the computer device may determine translation vectors for constructing a supercell of a user-specified size from the first data. The computer device according to an embodiment can determine translation vectors for constructing a supercell of a user-specified size from the first data. The translation vectors are vectors used to construct a supercell by repeatedly translating the unit cell by a given direction and distance. For example, if a user specifies a 2×2×2 supercell, the computer device can generate translation vectors by doubling each basis vector of the unit cell. If the basis vectors of the unit cell are a, b, and c, the translation vectors would be 2a, 2b, and 2c.

In step S420, the computer device according to an embodiment may construct a supercell composed of a plurality of unit cells by translationally moving the unit cell of the first data using the translation vectors. This is achieved by repeatedly translating the unit cell by the direction and magnitude of the translation vectors to generate adjacent unit cells. In the example of a 2×2×2 supercell, the computer device can generate a total of 8 unit cells by translating the unit cell by magnitudes of 0 and 1 in the a, b, and c directions, respectively. The generated unit cells share faces and form a continuous supercell.

In step S430, the computer device according to an embodiment may generate supercell structure data by merging identical atoms existing at the boundaries between adjacent unit cells within the supercell. The plurality of unit cells generated in the translation process of step S420 share boundaries, and identical atoms may exist redundantly on these boundaries. To identify these duplicate atoms, the computer device can calculate the remainder after dividing the coordinates of each atom by the basis vectors of the supercell. Atoms with the same remainder value can be considered identical atoms that map to each other through translational symmetry operations. By keeping only one of these identified duplicate atoms and removing the rest, the computer device can generate the second data, which is continuous and periodic supercell structure data. This second data includes the coordinate and species information of all atoms constituting the supercell.

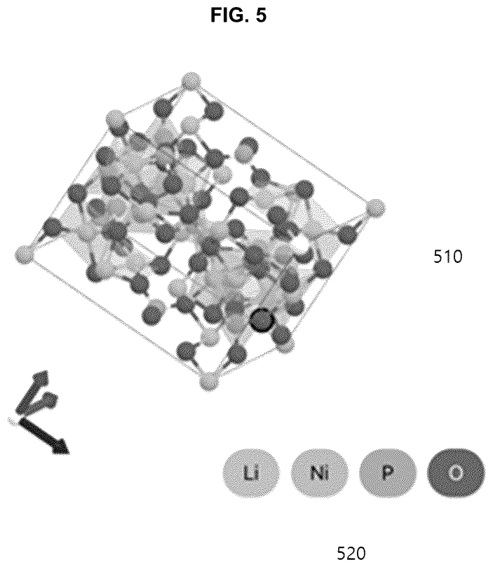

FIG. 5 is a diagram for explaining input data according to an embodiment of the present disclosure.

As shown in FIG. 5, the crystal structure 510 according to an embodiment visualizes the crystal structure of the input data in three dimensions. The crystal structure 510 may include various atoms, for example, Li, Ni, P, and O. These atoms can be distinguished based on a separate atom identifier 520.

The computer device can assign a unique integer identifier to each atom included in the second data according to the species of the atoms. This integer identifier may correspond to the atomic number or the order of the atom in the periodic table.

For example, Hydrogen (H) may be assigned 1, Helium (He) 2, Lithium (Li) 3, and so on. By doing this, the computer device can represent the species of each atom with a unique integer. The computer device can determine the size of a minimum-sized rectangular parallelepiped that encloses the region occupied by the supercell in three-dimensional space. This can be calculated by considering the coordinates of all atoms within the supercell, finding the smallest and largest x, y, and z coordinates to determine the two opposite vertices of the rectangular parallelepiped. This rectangular parallelepiped becomes the minimum-sized space containing the supercell.

The computer device can generate a three-dimensional grid by dividing the length of each edge of this rectangular parallelepiped by a user-specified resolution. For example, if the resolution is 0.5 Å and the lengths of the rectangular parallelepiped in the x, y, and z directions are 10 Å, 20 Å, and 30 Å, respectively, a three-dimensional grid with 20, 40, and 60 grid points in each direction is generated. This grid is a space that divides the supercell at regular intervals. The computer device can identify the presence or absence of an atom at each grid point of the three-dimensional grid. To do this, the computer device can calculate the Euclidean distance between a grid point and each atom in the second data, and if the minimum of these distances is less than or equal to a user-specified threshold (e.g., 0.1 Å), it can determine that an atom exists at that grid point. Through this process, the computer device can assign the presence or absence of an atom to each grid point.

The computer device can construct a three-dimensional grid by assigning the integer identifier of the corresponding atom to grid points where an atom is identified, and 0 to grid points where no atom is present. By doing so, the computer device can represent the atomic distribution of the supercell as a three-dimensional integer array. The computer device can convert this constructed three-dimensional grid into a four-dimensional tensor. Each grid point of the three-dimensional grid corresponds to three-dimensional spatial coordinates (x, y, z), and the value at each grid point is an integer identifier representing the atom species or 0. The computer device can separate this three-dimensional grid into several three-dimensional grids according to the atom species and stack them along a depth dimension to form a four-dimensional tensor. For example, if there are 3 types of atoms, the size of the four-dimensional tensor will be (3, x_size, y_size, z_size). In this case, the first dimension of the four-dimensional tensor corresponds to the atom species, and the remaining three dimensions correspond to the three-dimensional spatial coordinates.

The computer device can use this four-dimensional tensor as the input data for the neural network model. The four-dimensional tensor is in a format that can be directly used as input for a convolutional neural network and includes both atom species and position information. Through the process described above, the computer device can effectively convert the second data into a format of input data that can be processed by a neural network model.

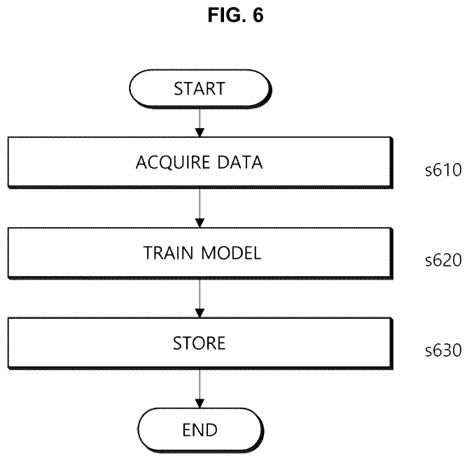

FIG. 6 is a diagram for explaining a learning operation of a computer device according to an embodiment of the present disclosure.

Referring to FIG. 6, in step S610, the computer device may acquire training data. The computer device can download crystal structure data in CIF format from a public crystal structure database. The computer device can receive crystal structure data in CIF format from a user. Also, the computer device can convert experimentally measured crystal structure data into CIF format. The computer device can convert the crystal structure data in CIF format into input data in the form of a four-dimensional tensor to generate training data.

In step S620, the computer device according to an embodiment may train the neural network model based on the training data.

The computer device can input the training data acquired and preprocessed in S610 into the neural network model in mini-batches, and calculate a loss function by comparing the model's output with the labels of the training data. The computer device can train the model by updating the weights of the neural network model using an optimization technique such as gradient descent in a direction that minimizes the calculated loss function. The computer device can control the learning speed and performance of the neural network model by adjusting hyperparameters such as the learning rate, mini-batch size, and number of training epochs. The computer device can evaluate the performance of the model on validation data at regular intervals during training and can stop the training by applying an early stopping technique if overfitting occurs.

In step S630, the computer device according to an embodiment may save the trained neural network model. The computer device can serialize the structure and trained weights of the trained neural network model and save them to a file.

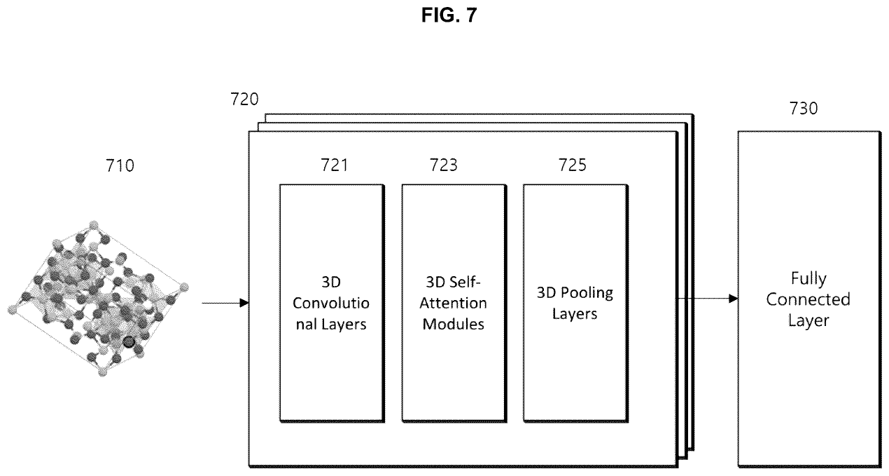

FIG. 7 is a block diagram illustrating a neural network model according to an embodiment of the present disclosure.

As shown in FIG. 7, input data 710 is used as input to the neural network and can be analyzed in subsequent layers to predict various physical properties.

The first neural network 720 may include 3D Convolutional Layers 721, 3D Self-Attention Modules 723, and 3D Pooling Layers 725.

The 3D convolutional layers 721 can effectively capture the spatial interactions between atoms to learn complex patterns and features within the crystal structure. This layer can apply convolutional filters to multi-dimensional input data. Convolution is applied by sliding a kernel over each part of the input data, and at each position, the element-wise product of the kernel and the data is calculated and then summed.

The 3D self-attention modules 723 can enable the network to focus on important information among the features extracted by the convolutional layers. The 3D self-attention modules 723 calculate the relationships between input features to evaluate the importance of each feature. In this process, three elements, ‘query’, ‘key’, and ‘value’, are used, and an attention score is generated by calculating the similarity between the query and the key.

The 3D pooling layers 725 can reduce the computational complexity of the network, prevent overfitting, and simultaneously reduce the size of the feature maps.

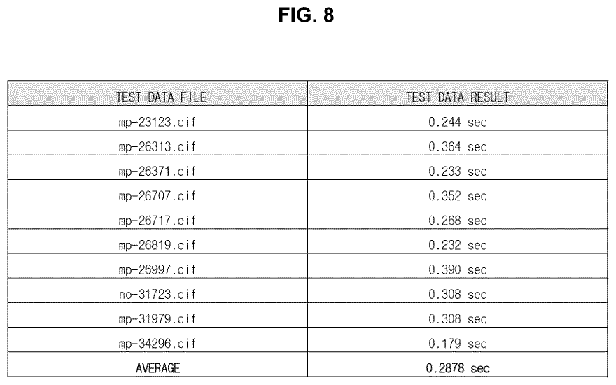

FIG. 8 is a table showing the time required for property estimation according to an embodiment of the present disclosure.

Specifically, it is the average response time measured after randomly extracting 10 sample data with Seed=42 from the Materials Project dataset, which is an open dataset. The average response time for the solution is 0.2878 sec, which is below the performance indicator of 120 sec.

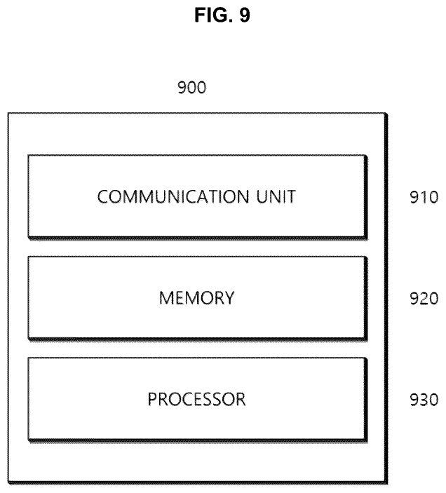

FIG. 9 is a block diagram illustrating a computer device according to an embodiment of the present disclosure.

The computer device is shown as being composed of a communication unit 910, a memory 920, and a processor 930, but is not necessarily limited to this configuration. The communication unit 910, memory 920, and processor 930 can each exist as a physically independent component.

The communication unit 910 performs functions for transmitting and receiving signals over a network. All or part of the communication unit 420 may be referred to as a transmitter, a receiver, or a transceiver. The communication unit 910 may refer to hardware and software components that enable the computer device to communicate with an external network. The communication unit 910 can support various communication protocols such as Ethernet, Wi-Fi, and Bluetooth, or provide a physical connection through an external connector such as an RJ45 jack or an antenna. A unique identifier called a MAC address is assigned within the adapter, which allows the computer device to be uniquely identified on the network.

The memory 920 can store various data for the overall operation of the computer device, such as programs for the processing or control by the processor 930.

The memory 920 can store a plurality of application programs that are executed, data for the operation of the computer device, and instructions. The memory 920 may be implemented as internal memory such as ROM and RAM included in the processor 930, or as a separate memory from the processor 930. According to an embodiment, the memory 920 can store a neural network and training data.

The processor 930 may be a component for controlling the computer device overall.

According to an embodiment, the processor 930 generates first data indicating a crystal structure from crystal structure description data, generates second data indicating an expanded supercell that considers the periodicity of the crystal structure based on the first data, converts the second data into input data in the form of a four-dimensional tensor with a first dimension for atom species and three additional dimensions representing three-dimensional spatial coordinates, inputs the input data into a neural network model based on a convolutional neural network and an attention mechanism to extract features, and estimates material properties from the extracted features.

According to an embodiment, the processor 930 can parse unit cell parameters, symmetry operations, and atomic coordinates of the crystal structure from a CIF, construct basis vectors of the unit cell from the parsed unit cell parameters, apply the parsed symmetry operations to the basis vectors to generate symmetry-equivalent points within the unit cell, and calculate atomic positions within the unit cell using the parsed atomic coordinates and the generated symmetry-equivalent points to generate unit cell structure data.

According to an embodiment, the processor 930 can determine translation vectors for constructing a supercell of a user-specified size from the first data, construct a supercell composed of a plurality of unit cells by translationally moving the unit cell of the first data using the translation vectors, and generate supercell structure data by merging identical atoms existing at the boundaries between adjacent unit cells within the supercell.

According to an embodiment, the processor 930 can assign a unique integer identifier to each atom included in the second data according to the species of the atoms.

According to an embodiment, the processor 930 can determine the size of a minimum-sized rectangular parallelepiped that encloses the region occupied by the supercell in three-dimensional space, generate a three-dimensional grid by dividing the length of each edge of the rectangular parallelepiped by a user-specified resolution, identify an atom at each grid point of the three-dimensional grid, assign the integer identifier of the corresponding atom if an atom is identified or 0 if no atom is identified, convert the three-dimensional grid with assigned integer identifiers or 0 into a four-dimensional tensor, and use the four-dimensional tensor as input data for the neural network model.

According to an embodiment, the processor 930 can calculate the distance between a grid point and each atom included in the second data, and determine that an atom is identified at the grid point if the smallest of the calculated distances is less than or equal to a user-specified threshold.

Specifically, the processor 930 can control the operation of the computer device using various programs stored in the memory 920 of the computer device. The processor 930 may include a CPU, RAM, ROM, a system bus, etc. The processor 930 may be implemented as a single CPU or a plurality of CPUs (or DSPs, SoCs). As an example, the processor 930 may be implemented as a digital signal processor (DSP), a microprocessor, or a TCON (Time controller). However, it is not limited to these, and may include one or more of a central processing unit (CPU), a Micro Controller Unit (MCU), a micro processing unit (MPU), a controller, an application processor (AP), or a communication processor (CP), an ARM processor, or be defined by such terms. Furthermore, the processor 930 may be implemented as a System on Chip (SoC) with an embedded processing algorithm, a large scale integration (LSI), or in the form of a Field Programmable Gate Array (FPGA). Moreover, the processor 930 may include a Neural Processing Unit (NPU), a Graphics Processing Unit (GPU), and a Tensor Processing Unit (TPU).

Although the embodiments have been described by limited embodiments and drawings as described above, various modifications and variations are possible from the above description by those skilled in the art. For example, appropriate results can be achieved even if the described technologies are performed in a different order from the described method, and/or the components of the described system, structure, device, circuit, etc., are combined or integrated in a different form from the described method, or replaced or substituted by other components or their equivalents.

Therefore, other implementations, other embodiments, and equivalents to the claims are intended to be included within the scope of the following claims.

What is claimed is:

1. A computer-implemented method comprising:
generating first data indicating a crystal structure of a material from crystal-structure description data;
based on the first data and periodicity of the crystal structure, generating second data indicating an expanded supercell;
generating input data from the second data by (i) defining a three-dimensional spatial region occupied by the supercell as a three-dimensional grid having a predetermined resolution, (ii) identifying, for each grid point of the three-dimensional grid, a species of an atom located at the grid point, and (iii) forming the input data as a four-dimensional tensor having a first dimension corresponding to atom species and three remaining dimensions corresponding to three-dimensional grid coordinates; extracting features by inputting the input data to a neural network model based on convolutional neural networks and an attention mechanism; and
estimating, from the extracted features, at least one physical property of the material.

2. The method of claim 1, wherein the crystal-structure description data is in a Crystallographic Information File (CIF) format.

3. The method of claim 2, wherein generating the first data comprises:
parsing, from the CIF, unit-cell parameters, symmetry operations, and atomic coordinates;
constructing basis vectors of a unit cell from the parsed unit-cell parameters;
applying the parsed symmetry operations to the basis vectors to generate symmetry-equivalent points in the unit cell; and
computing atomic positions in the unit cell using the parsed atomic coordinates and the generated symmetry-equivalent points to produce unit-cell structure data,
wherein the unit-cell structure data constitutes the first data.

4. The method of claim 1, wherein generating the second data comprises:
determining translation vectors to construct a supercell of a user-specified size from the first data;
translationally replicating the unit cell using the translation vectors to construct the supercell including a plurality of unit cells; and
merging identical atoms located at boundaries between adjacent unit cells within the supercell to produce supercell structure data,
wherein the supercell structure data constitutes the second data.

5. The method of claim 1, wherein generating the input data further comprises assigning, to each atom included in the second data, a unique integer identifier according to an atom species.

6. The method of claim 5, further comprising:
determining dimensions of a minimum axis-aligned rectangular parallelepiped enclosing the spatial region occupied by the supercell;
dividing each edge length of the rectangular parallelepiped by a user-specified resolution to generate the three-dimensional grid;
identifying, for each grid point of the three-dimensional grid, whether an atom is present;
assigning, when an atom is identified at a grid point, the integer identifier of the atom to the grid point and, when no atom is identified, assigning a value of zero to the grid point;
converting the resulting three-dimensional grid of integer identifiers or zeros into the four-dimensional tensor; and
providing the four-dimensional tensor as the input data to the neural network model,
wherein a first dimension of the four-dimensional tensor corresponds to atom species and remaining three dimensions correspond to locations in three-dimensional space.

7. The method of claim 6, wherein the integer identifier corresponds to an atomic number or to an ordering of elements in the periodic table.

8. The method of claim 6, wherein identifying whether an atom is present at each grid point comprises:
computing distances between the grid point and respective atoms included in the second data; and
determining that an atom is identified for the grid point when a smallest one of the computed distances is less than or equal to a user-specified threshold.

9. The method of claim 6, wherein the resolution of the three-dimensional grid is user-determined.

10. The method of claim 1, wherein the neural network model comprises a plurality of convolutional layers, a plurality of pooling layers, and at least one self-attention module.

11. The method of claim 1, wherein the physical property comprises at least one of activation energy, Young's modulus, and interfacial energy.

12. The method of claim 1, wherein the material comprises at least one of a cathode active material, an anode active material, an electrolyte, and a separator of a lithium-metal secondary battery.

13. A non-transitory computer-readable medium storing instructions that, when executed by one or more processors, cause the one or more processors to:
receive, as input data, a four-dimensional tensor including three-dimensional atomic coordinate information of a crystal structure of a material whose physical property is to be predicted,
wherein a first dimension of the four-dimensional tensor represents atom species, three remaining dimensions represent location coordinates on a three-dimensional grid obtained by discretizing a spatial region occupied by the crystal structure, and input the four-dimensional tensor to a three-dimensional convolutional neural network to extract features, the three-dimensional convolutional neural network including a plurality of three-dimensional self-attention modules, and
wherein each three-dimensional self-attention module comprises: (i) three three-dimensional convolutional layers configured to transform an input feature map into a query (Q), a key (K), and a value (V); (ii) a layer configured to compute an inner product between the query and the key to generate attention weights; and (iii) a layer configured to multiply the attention weights with the value to produce a weighted feature map; and predict, from the extracted features, at least one physical property of the material.
